(19) United States
(12) Patent Application Publication
(10) Pub. No.: US 2026/0220237 A1
(43) Pub. Date: Jul. 30, 2026

(54) INFORMATION PROCESSING APPARATUS, INFORMATION PROCESSING METHOD, AND NON-TRANSITORY RECORDING MEDIUM

(71) Applicant: NEC Corporation, Tokyo (JP)

(72) Inventors: Masato SASAKI, Tokyo (JP); Ryoma OAMI, Tokyo (JP); Kosuke YOSHIMI, Tokyo (JP); Ryo YAMAKABE, Tokyo (JP); Megumi HASHIMOTO, Tokyo (JP); Shinji OOMINATO, Tokyo (JP)

(73) Assignee: NEC Corporation, Tokyo (JP)

(21) Appl. No.: 19/143,006

(22) PCT Filed: Jan. 11, 2023
(86) PCT No.: PCT/JP2023/000413

§ 371 Date: Jun. 25, 2025

(51) Int. Cl. G06F 21/32

(52) U.S. Cl. CPC G06F 21/32

(57) ABSTRACT

An information processing apparatus including: an authentication unit that performs an authentication on a target using biometric information of the target; a storing unit that stores target information in a storing apparatus in association with the target, wherein the target information including the biometric information and authentication information related to the authentication using the biometric information; and an output unit that outputs body information related to a body of the target, which is estimated using two or more pieces of the target information of the target stored in the storing apparatus.

### TECHNICAL FIELD

This disclosure relates to the technical field of information processing apparatus, information processing methods, and recording media.

### BACKGROUND ART

Patent Literature 1 describes a technology in which a first part acquires biometric information of a human body, uses biological authentication results based on the acquired biometric information, transmits the acquired biometric information to a second part, and the second part processes the biometric information transmitted from the first part as health management information of the human body.

Patent Literature 2 describes an apparatus comprising an antenna section with a MIMO radar flat antenna arranged on the front surface and a display section with a display panel, wherein the flat antenna and the display panel are facing forward from the front surface, and the antenna section is rotatable relative to the display section so that the flat antenna faces the rear surface of the display section opposite to the display panel. The display unit is rotatably coupled to the antenna unit, enabling detection of biological signals on the front side of the front surface and biological signals on the rear side facing the front surface.

Patent Literature 3 describes a technology in which, at an entrance gate, when body temperature information, etc. is read by a code reader from an information code displayed on a mobile terminal carried by a user, the code reader determines whether or not the user who held the mobile terminal in front of the code reader is allowed to enter based on the reading result, and a warning light changes to a predetermined lighting state according to the determination result.

### CITATION LIST

### Patent Literature

Patent Literature 1: JP2013-257906A
Patent Literature 2: JP7086441B
Patent Literature 3: JP2022-110361A

### SUMMARY

It is an example object of this disclosure to provide an information processing apparatus, an information processing method, and a recording medium that are intended to improve the techniques/technologies disclosed in Citation List.

### Solution to Problem

An information processing apparatus according to an example aspect includes:

An information processing apparatus including:
an authentication unit that performs an authentication on a target using biometric information of the target;
a storing unit that stores target information in a storing apparatus in association with the target, wherein the target information including the biometric information and authentication information related to the authentication using the biometric information; and
an output unit that outputs body information related to a body of the target, which is estimated using two or more pieces of the target information of the target stored in the storing apparatus.

An information processing method according to an example aspect includes: performing an authentication on a target using biometric information of the target; storing target information in a storing apparatus in association with the target, wherein the target information including the biometric information and authentication information related to the authentication using the biometric information; and outputting body information related to a body of the target, which is estimated using two or more pieces of the target information of the target stored in the storing apparatus.

A recording medium according to an example aspect is a recording medium on which a computer program that allows a computer to execute an information processing method is recorded, the information processing method including: performing an authentication on a target using biometric information of the target; storing target information in a storing apparatus in association with the target, wherein the target information including the biometric information and authentication information related to the authentication using the biometric information; and outputting body information related to a body of the target, which is estimated using two or more pieces of the target information of the target stored in the storing apparatus.

### BRIEF DESCRIPTION OF DRAWINGS

FIG. 1 is a block diagram illustrating a configuration of an information processing apparatus according to this disclosure.

FIG. 2 is a block diagram illustrating a configuration of an information processing apparatus according to this disclosure.

FIG. 3 is a flowchart illustrating a flow of an information processing operation of the information processing apparatus according to this disclosure.

FIG. 4 is a block diagram illustrating a configuration of an information processing apparatus according to this disclosure.

FIG. 5 is a block diagram illustrating a configuration of an information processing apparatus according to this disclosure.

FIG. 6 is a block diagram illustrating a configuration of an information processing apparatus according to this disclosure.

FIG. 7 is a flowchart illustrating a flow of an information processing operation of the information processing apparatus according to this disclosure.

FIG. 8 is a block diagram illustrating a configuration of an information processing apparatus according to this disclosure.

FIG. 9 is a flowchart illustrating a flow of an information processing operation of the information processing apparatus according to this disclosure.

FIG. 10 is a block diagram illustrating a configuration of an information processing apparatus according to this disclosure.

FIG. 11 is a block diagram illustrating a configuration of an information processing apparatus according to this disclosure.

FIG. 12 is a block diagram illustrating a configuration of an information processing apparatus according to this disclosure.

FIG. 13 is a flowchart illustrating a flow of an information processing operation of the information processing apparatus according to this disclosure.

### DESCRIPTION OF EXAMPLE EMBODIMENTS

The following describes embodiments of the information processing apparatus, the information processing method, and the recording medium with reference to the drawings.

### 1: First Example Embodiment

A first embodiment of an information processing apparatus, information processing method, and recording medium is described. The first embodiment of the information processing apparatus, information processing method, and recording medium will be described below using an information processing apparatus 1 to which the first embodiment of the information processing apparatus, information processing method, and recording medium is applied.

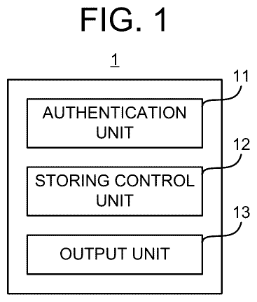

FIG. 1 is a block diagram showing the configuration of the information processing apparatus 1 according to the first embodiment. As shown in FIG. 1, the information processing apparatus 1 includes an authentication unit 11, a storing control unit 12, and an output unit 13.

The authentication unit 11 authenticates a target person using biometric information of the target person. The storing control unit 12 stores target information, including biometric information and authentication information related to the authentication using the biometric information, in association with the target person. The output unit 13 outputs body information related to a body of the target person, which is estimated using two or more pieces of the target information stored in the storing control unit 12.

### 1-2: Technical Effects of the Information Processing Apparatus 1

The information processing apparatus 1 according to the first embodiment can estimate the body of the target person from the authentication information related to two or more authentications and the biometric information used for said authentication, and output the body information related to the body of the target person.

### 2: Second Example Embodiment

Next, a second embodiment of the information processing apparatus, information processing method, and recording medium will be described. The second embodiment of the information processing apparatus, information processing method, and recording medium will be described using an information processing apparatus 2 to which the second embodiment of the information processing apparatus, information processing method, and recording medium is applied.

### 2-1: Configuration of the Information Processing Apparatus 2

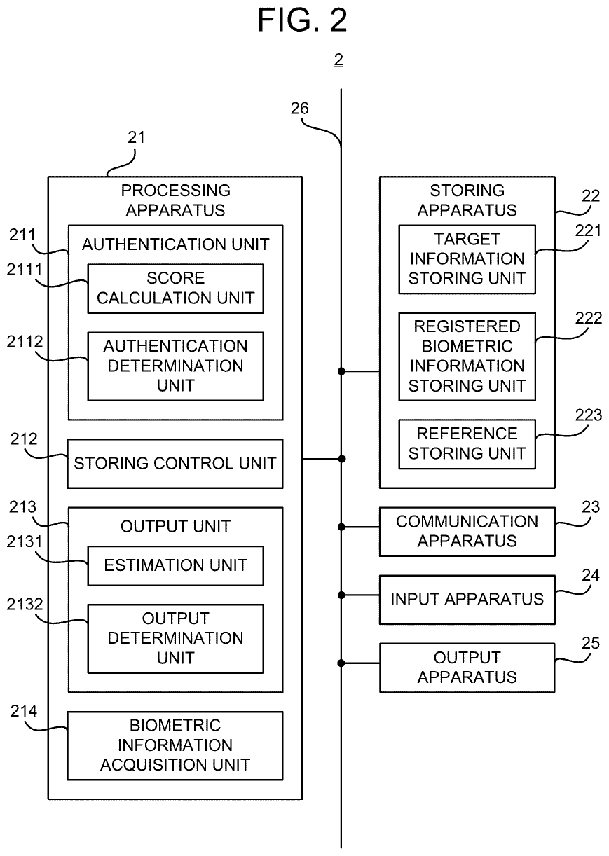

5 FIG. 2 is a block diagram showing the configuration of the information processing apparatus 2 according to the second embodiment. As shown in FIG. 2, the information processing apparatus 2 includes a processing apparatus 21 and a storing apparatus 22. Furthermore, the information processing apparatus 2 may include a communication apparatus 23, an input apparatus 24, and the output apparatus 25. However, the information processing apparatus 2 may not include at least one of the communication apparatus 23, the input apparatus 24, and an output apparatus 25. The processing apparatus 21, the storing apparatus 22, the communication apparatus 23, the input apparatus 24, and the output apparatus 25 may be connected via a data bus 26.

The processing apparatus 21 may be, for example, a CPU (Central Processing Unit), a GPU (Graphics Processing Unit), and/or an FPGA (Field Programmable Gate Array). The processing apparatus 21 reads a computer program. For example, the processing apparatus 21 may read a computer program stored in the storing apparatus 22. For example, the processing apparatus 21 may read a computer program stored in a computer-readable and non-temporary recording medium using a recording medium reading apparatus (e.g., the input apparatus 24 described later) provided in the information processing apparatus 2. The processing apparatus 21 may acquire (i.e., download or load) a computer program from an apparatus not shown, which is disposed outside the information processing apparatus 2 via the communication apparatus 23 (or other communication apparatus). The processing apparatus 21 executes the loaded computer program. As a result, logical function blocks for executing operations to be performed by the information processing apparatus 2 are realized within the processing apparatus 21. In other words, the processing apparatus 21 functions as a controller capable of realizing logical functional blocks for executing the operations (i.e., processing) that the information processing apparatus 2 should perform.

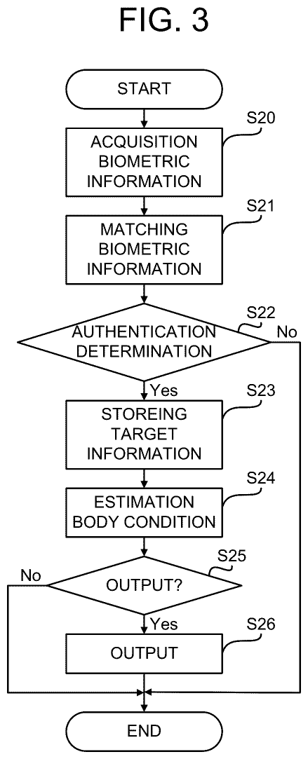

FIG. 2 shows an example of logical functional blocks realized within the processing apparatus 21 for executing information processing operations. As shown in FIG. 2, the processing apparatus 21 includes an authentication unit 211, which is a specific example of “an authentication unit” described in the Supplementary Note described later, a storing control unit 212, which is a specific example of “a storing unit” described in the Supplementary Note described later, an output unit 213, which is a specific example of “an output unit” described in the Supplementary Note described later, and a biometric information acquisition unit 214. The authentication unit 211 includes a score calculation unit 2111 and an authentication determination unit 2112. Additionally, the output unit 213 includes an estimation unit 2131 and an output determination unit 2132. However, within the processing apparatus 21, at least one of the storing control unit 212 and the biometric information acquisition unit 214 may be omitted. Furthermore, the authentication unit 211 may optionally include the score calculation unit 2111. Furthermore, the output unit 213 need not necessarily include at least one of the estimation unit 2131 and the output determination unit 2132. The output unit 213 may include the estimation unit 2131 and need not include the output determination unit 2132. In this case, the function of the output determination unit 2132 described below may be realized separately from the output unit 213 within the information processing apparatus 2. Alternatively, the function of the output determination unit 2132 may be implemented in a cloud external to the information processing apparatus 2. Furthermore, the output unit 213 may have the estimation unit 2131 and may have the output determination unit 2132. In this case, the function of the estimation unit 2131 described later may be implemented separately from the output unit 213 within the information processing apparatus 2. Alternatively, the functions of the estimation unit 2131 may be implemented in a cloud external to the information processing apparatus 2. Furthermore, the output unit 213 may not include the estimation unit 2131 or the output determination unit 2132. In this case, at least one of the functions of the later-described the estimation unit 2131 and the output determination unit 2132 may be implemented separately from the output unit 213 within the information processing apparatus 2. Alternatively, at least one of the functions of the later-described the estimation unit 2131 and the output determination unit 2132 may be implemented in a cloud external to the information processing apparatus 2. In case the output unit 213 does not include the estimation unit 2131 and the output determination unit 2132, the output unit 213 may be a mechanism that acquires the estimated body information to be output and outputs the body information. The details of the operation of the authentication unit 211, the storing control unit 212, the output unit 213, and the biometric information acquisition unit 214 will be explained later with reference to FIG. 3.

The storing apparatus 22 is capable of storing desired data. For example, the storing apparatus 22 may temporarily store the computer program executed by the processing apparatus 21. The storing apparatus 22 may temporarily store data that the processing apparatus 21 temporarily uses in case of executing a computer program. The storing apparatus 22 may store data that the information processing apparatus 2 stores long-term. Note that the storing apparatus 22 may be RAM (Random Access Memory), ROM (Read Only Memory), a hard disk apparatus, an optical magnetic disk apparatus, an SSD (Solid State Drive), and a disk array apparatus. In other words, the storing apparatus 22 may include a non-temporary recording medium. The storing apparatus 22 may include a target information storing unit 221, a registered biometric information storing unit 222, and a reference storing unit 223, which are specific examples of “a storing apparatus” described in the Supplementary Note described later. The storing control unit 212, which is a specific example of “the storing unit” described in the Supplementary Note described later, may store information in the target information storing unit 221, which is a specific example of “the storing apparatus” described in the Supplementary Note described later. However, the target information storing unit 221, the registered biometric information storing unit 222, and the reference storing unit 223 need not be realized within the storing apparatus 22. In case any of the storing units the target information storing unit 221, the registered biometric information storing unit 222, and the reference storing unit 223 are not realized within the storing apparatus 22, the storing units may be realized in an apparatus external to the information processing apparatus 2. In this case, the processing apparatus 21 may transmit and receive information with the storing units realized in the external apparatus, for example, via the communication apparatus 23 described later. Details of the target information storing unit 221, the registered biometric information storing unit 222, and the reference storing unit 223 will be explained later with reference to FIG. 3 and Table 1.

The communication apparatus 23 is capable of communicating with an external apparatus of the information processing apparatus 2 via a communication network (not shown). The communication apparatus 23 may be a communication interface based on standards such as Ethernet (registered trademark), Wi-Fi (registered trademark), Bluetooth (registered trademark), or USB (Universal Serial Bus).

The input apparatus 24 is an apparatus that accepts information input to the information processing apparatus 2 from outside the information processing apparatus 2. For example, the input apparatus 24 may include an operation apparatus (e.g., at least one of a keyboard, a mouse, and a touch panel) that can be operated by an operator of the information processing apparatus 2. For example, the input apparatus 24 may include a reading apparatus that can read information recorded as data on a recording medium that can be attached to the information processing apparatus 2.

The output apparatus 25 is an apparatus that outputs information to the outside of the information processing apparatus 2. For example, the output apparatus 25 may output information as images. In other words, the output apparatus 25 may include a display apparatus (a so-called display) capable of displaying images indicating the information to be output. For example, the output apparatus 25 may output information as sound. In other words, the output apparatus 25 may include a sound output apparatus (i.e., a speaker) capable of outputting sound. For example, the output apparatus 25 may output information to paper. In other words, the output apparatus 25 may include a printing apparatus (i.e., a printer) capable of printing desired information on paper.

### 2-2: Application Scenes of the Information Processing Apparatus 2

The information processing apparatus 2 according to this embodiment may be applied to situations where biometric authentication using biometric information of a target person is performed. The information processing apparatus 2 may be applied to situations where biometric authentication using images such as iris authentication and face authentication is performed. In addition, the information processing apparatus 2 may be applied to situations where biometric authentication such as voice authentication using a microphone, ear acoustic authentication using earphones, fingerprint authentication, and vein authentication is performed. The information processing apparatus 2 may be applied to situations where biometric authentication of the target person is performed on a daily basis, such as entry and exit to restricted areas where only authenticated the target person are allowed to enter, such as offices and apartment buildings.

### 2-3: Information Processing Operations Performed by the Information Processing Apparatus 2

Referring to FIG. 3, the information processing operations performed by the information processing apparatus 2 will be described. FIG. 3 is a flowchart showing the flow of information processing operations performed by the information processing apparatus 2. The information processing apparatus 2 may start information processing operations in response to instructions to perform authentication by the target person.

As shown in FIG. 3, the biometric information acquisition unit 214 acquires biometric information (step S20). For example, in case the information processing apparatus 2 performs iris authentication, the biometric information acquisition unit 214 may acquire eye images that includes an iris. In this case, the biometric information acquisition unit 214 may acquire the eye images captured by an infrared camera. Alternatively, the biometric information acquisition unit 214 may acquire the eye images captured by a visible light camera. In addition, in case the information processing apparatus 2 performs face authentication, the biometric information acquisition unit 214 may acquire face images that includes a face. In this case, the biometric information acquisition unit 214 may acquire the face images captured by a visible light camera. Alternatively, the biometric information acquisition unit 214 may acquire the face images captured by an infrared camera. In addition, in case the information processing apparatus 2 performs multi-modal authentication including iris authentication and face authentication, the biometric information acquisition unit 214 may acquire the eye images and the face images.

The score calculation unit 2111 matches the biometric information of the target person with the biometric information registered in the registered biometric information storing unit 222 (step S21). The score calculation unit 2111 may match features extracted from the biometric information of the target person with features extracted from the biometric information registered in the registered biometric information storing unit 222. The score calculation unit 2111 calculates an authentication score.

The authentication determination unit 2112 determines whether the authentication was successful or not using the matching result acquired by the score calculation unit 2111 (step S22). The authentication determination unit 2112 may determine whether the authentication was successful or not depending on whether the authentication score acquired as the matching result exceeds a predetermined threshold value. In other words, the authentication unit 211 authenticates the target person using the biometric information of the target person. The authentication determination unit 2112 may determine whether or not to allow entry to an office, apartment building, etc. based on the authentication result.

In case authentication is unsuccessful (step S22: No), the information processing operation ends. In this case, the target person may perform the instructions to perform authentication again for the information processing apparatus 2.

In case authentication is successful (step S22: Yes), the storing control unit 212 stores the target information, including the biometric information and the authentication information related to the authentication using the biometric information, in the target information storing unit 221, that be associated with the target person (step S23). The storing control unit 212 stores the target information in the target information storing unit 221, that be associated with the target person whose identity has been identified. The storing control unit 212 may store at least one of the eye image and the face image as the biometric information in the target information storing unit 221. The storing control unit 212 may store the authentication information including information indicating the date and time when the authentication was performed and the authentication score of the performed authentication in the target information storing unit 221.

The estimation unit 2131 estimates the body information of the target person using two or more pieces of the target information related to the authentication performed on different dates and times of the target person stored in the target information storing unit 221 (step S24). The estimation unit 2131 may estimate the body information of the target person using at least one past target information and current target information. The estimation unit 2131 may estimate the body information of the target person by comparing at least one the past target information and the current target information. The estimation unit 2131 may estimate the body information of the target person based on the time series changes in the biometric information stored in the target information storing unit 221. The estimation unit 2131 may estimate the body information of the target person based on the time series changes in the authentication scores stored in the target information storing unit 221.

The body information may include health information indicating health condition of the target person. The health information may include information indicating signs of illness. The estimation unit 2131 may detect the signs of illness. In case the estimation unit 2131 detects the signs of illness, it may recommend visiting a medical institution. The health information may include information indicating a recommendation to visit the medical institution.

The estimation unit 2131 may estimate body condition, including body abnormalities, based on at least one of the biometric information acquired on different dates and times and the authentication score. The authentication is performed daily, and changes in the authentication score are often related to body changes. The estimation unit 2131 may detect daily changes in the body condition of the target person by using information that can be acquired through the authentication. The estimation unit 2131 may suspect the signs of illness in case the authentication score declines after observation over time. Furthermore, the estimation unit 2131 may acquire information indicating fitness status, such as the number of steps taken, and use it to estimate the body information. The estimation unit 2131 may acquire information indicating the fitness status, such as the number of steps taken, from apparatus carried or worn by the target person, such as smartphones and smart watches.

The estimation unit 2131 may detect whether a change in the body is sudden or gradual. For example, in case a sudden change in the body is detected, the estimation unit 2131 may estimate that it is a pathological change. For example, in case the body changes in a single day, the estimation unit 2131 may estimate that it is a pathological change. In case a gradual change in the body is detected, the estimation unit 2131 may assume that the change is not pathological. For example, in case the change occurred over a long period of time, it may be assumed to be a natural change due to aging. In case the change is due to poor health on a particular day and only the following day is in a special state, the estimation unit 2131 may not assume that the change is pathological.

The estimation unit 2131 may have multiple methods for estimating body condition based on information included in images. The method for estimating body condition from images and authentication scores in time series using the estimation unit 2131 will be described later.

The estimation unit 2131 may make estimates regarding specified items based on information stored in the target information storing unit 221. The items may be subjects of estimates by the estimation unit 2131. The items may be detection subjects for which a specified state is detected by the estimation unit 2131. The items may be subjects for detecting abnormalities. The items may be related to the signs of illness or unrelated to the signs of illness. Whether or not they are the signs of illness may be determined based on the level described later. The items may be arbitrarily defined. Body parts may be defined as the items. For example, eye area or mouth area may be defined as the items. Additionally, the items may include body temperature or heart rate.

The estimation unit 2131 may determine that an abnormality exists in case a predetermined state is detected in the image. The predetermined state may be a difference between the left and right sides of a predetermined part, or a time series change in that difference. For example, even if there is a difference between the left and right sides, in case that difference continues for a long period of time, the estimation unit 2131 may determine that the difference is a characteristic of the target person.

The output determination unit 2132 determines whether or not to output the estimation result (step S25). The output determination unit 2132 may determine whether or not to notify the target person of the estimation result. The output determination unit 2132 may compare the estimation result based on at least one of the image acquired by the estimation unit 2131 and the authentication score with reference information to determine whether or not to output the estimation result. The output determination unit 2132 may make a determination based on the degree of difference between the estimated result and the reference information. The estimation unit 2131 may calculate a value as the estimated result for a predetermined item. The reference information may indicate a value or a range. The estimation by the estimation unit 2131 may be rephrased as the detection of the detection target for the determination by the output determination unit 2132.

The reference storing unit 223 stores information used by the output determination unit 2132 for determination. The output determination unit 2132 may refer to the reference storing unit 223 to determine the level of an item. The output determination unit 2132 may determine whether to output based on comparison with information stored in the reference storing unit 223. The reference storing unit 223 may store, for example, as shown in Table 1 below, a range of levels for each item.

TABLE 1
[The reference storing unit 223]

| Item | Level | Range |
|---|---|---|
| A | 1 | 1AL to 1AH |
| A | 2 | 2AL to 2AH |
| A | 3 | 3AL to 3AH |
| A | 4 | 4AL to 4AH |
| A | 5 | 5AL to 5AH |
| B | 1 | 1BL to 1BH |
| B | 2 | 2BL to 2BH |

.
.
.

The reference storing unit 223 may store level ranges divided into five levels, such as “1,” “2,” “3,” “4,” and “5,” for each item. The level ranges may be the ranges of differences between a value as the estimated result and a reference value. Level “1” may indicate a level where there is a mild abnormality but no particular problem and no attention is required. Level ‘2’ may indicate a level where there is a mild abnormality but no particular problem. The level ‘2’ may indicate a level where notification is performed for individuals who wish to be notified even if there is a mild abnormality, unlike the level ‘1.’ Levels ‘1’ and ‘2’ may be understood as safe levels. Level “3” may indicate a level where there is a high possibility that some disease is progressing. The level ‘3’ may be understood as a level requiring caution. The level ‘3’ may be a level where lifestyle improvements are recommended. The level ‘3’ may be a level where, depending on the item, a visit to a medical institution is recommended. Level ‘4’ may be a level where a visit to a medical institution is recommended. Level ‘5’ may be a level where immediate medical attention is required.

The output determination unit 2132 may determine in what level range which the difference between the value as the estimated result and the reference value falls. In the example shown in Table 1 above, the reference storing unit 223 indicates that the range for level 1 of item A is “1AL” to “1AH,” the range for level 2 of item A is “2AL” to “2AH,” the range for level 3 of item A is “3AL” to “3AH,” the range for level 4 of item A is “4AL” to “4AH,” and the range for level 5 of item A is “5AL” to “5AH.” In addition, the range of level 1 for item B is “1BL” to “1BH,” and the range of level 2 for item B is “2BL” to “2BH.” In this case, the output determination unit 2132 may determine that item A is level 1 in case the difference between the value as estimated result for item A and the reference value is within the range of “1AL” to “1AH.” Furthermore, the output determination unit 2132 may determine that item B is level 2 in case the difference between the value as the estimated result for item B and the reference value is within the range of “2BL” to “2BH.”

The reference information for each item may be a value or range indicating normalcy for that item. The reference information for each item may be determined from the past changes in the body information of multiple individuals who were determined to be abnormal for the same item at a medical institution. In other words, a model of individuals with a high probability of developing the disease in the future may be formed from information of multiple individuals who were determined to be abnormal for the same item at a medical institution. Similarly, a model of individuals with a low probability of developing the disease in the future may be formed from information of multiple individuals who were not determined to be abnormal for the same item at a medical institution. The reference information for each item may be determined from these formed models.

The output determination unit 2132 may determine whether or not to output according to the level. That is, even if the estimation unit 2131 detects a difference from the reference information, in case the output determination unit 2132 determines that the difference is not sufficient to output, it may not output.

In case it is determined that output should be made (step S25: Yes), the output unit 213 outputs the body information related to the body of the target person (step S26). The output unit 213 may transmit the body information to a terminal apparatus that can be used by the target person and notify the target person. The terminal apparatus may be a personal computer, a smartphone, or other apparatus that can be connected to the information processing apparatus 2 via the internet, for example. The output unit 213 may output a text explaining the level for the item along with related images, such as images that serve as the basis for the estimation.

In case the level is “3” or higher for any item, the output unit 213 may notify the target person of the health abnormality. In case the signs of illness are detected, the output unit 213 may output countermeasures corresponding to the signs of the illness. The countermeasures may include visiting a medical institution. In addition, in case the level for each item is “2,” the output unit 213 may notify the target person of their health condition.

### Modification: Determination According to the Target Person

In the modification, a required notification storing unit 224 may be implemented within the storing apparatus 22. The required notification storing unit 224 may include information indicating the level at which the target person wishes to be notified of the body information. The output determination unit 2132 may make a determination appropriate for the target person by referring to the required notification storing unit 224. The required notification storing unit 224 may store information indicating whether notification of the body information is required at the applicable level for each item, as illustrated in the following Table 2.

TABLE 2
[The required notification storing unit 224]

| Target Person | Item | Level | Notification Required |
|---|---|---|---|
| P1 | A | 1 | Not Required |
| P1 | A | 2 | Not Required |
| P1 | A | 3 | Required |
| P1 | A | 4 | Required |
| P1 | A | 5 | Required |
| P1 | B | 1 | Not Required |
| P1 | B | 2 | Required |

.
.
.

| | | | |
|---|---|---|---|
| P2 | A | 1 | Not Required |
| P2 | A | 2 | Required |

.
.
.

The required notification storing unit 224 may store information indicating whether notification of the body information is required at each level, such as “1,” “2,” “3,” “4,” and “5,” for each target person and each item. In the case illustrated in Table 2 above, the required notification storing unit 224 may store information indicating that notification is not required in case the level of item A is 1 or 2 for target person P1, and that notification is required in case the level of item A is 3 to 5. Additionally, the required notification storing unit 224 stores information indicating that notification is not required for target person P1 in case the level of item B is 1, and that notification is required in case the level of item B is 2. Furthermore, the required notification storing unit 224 stores information indicating that notification is not required for target person P2 in case the level of item A is 1, and that notification is required in case the level of item A is 2.

### 2-4: Technical Effects of the Information Processing Apparatus 2

The information processing apparatus 2 according to the second embodiment uses images of the target person taken during the authentication for purposes other than the authentication. The information processing apparatus 2 can utilize images taken during the authentication and the authentication scores acquired during the authentication for healthcare purposes. The information processing apparatus 2 can accurately grasp condition of the target person because it can grasp daily condition of the target person, which is more accurate than regular condition assessments conducted on a monthly basis, for example.

The information processing apparatus 2 notifies the target person in case an abnormality occurs, enabling early detection of health abnormalities and allowing the target person to take appropriate action. The target person can identify health condition or signs of illness that they may not have noticed on their own through the notification from the body information. Furthermore, this can contribute to the promotion of health of the target person.

### 3: Third Example Embodiment

Next, the third embodiment of the information processing apparatus, information processing method, and recording medium will be described. In the following, the third embodiment of the information processing apparatus, information processing method, and recording medium will be described using an information processing apparatus 3 to which the third embodiment of the information processing apparatus, information processing method, and recording medium is applied.

### 3-1: Configuration of the Information Processing Apparatus 3

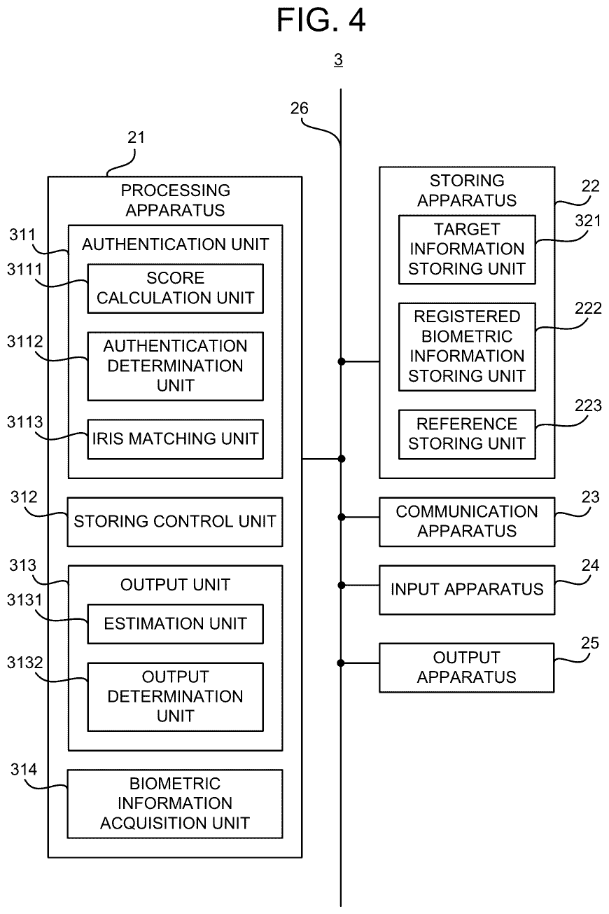

As shown in FIG. 4, the information processing apparatus 3 according to the third embodiment is similar to the information processing apparatus 2 according to the second embodiment and includes the processing apparatus 21 and the storing apparatus 22. Furthermore, the information processing apparatus 3 according to the third embodiment may include the communication apparatus 23, the input apparatus 24, and the output apparatus 25 similar to the information processing apparatus 2 according to the second embodiment. However, the information processing apparatus 3 may not include at least one of the communication apparatus 23, the input apparatus 24 and the output apparatus 25. The third embodiment of the information processing apparatus 3 differs from the information processing apparatus 2 according to the second embodiment in that an authentication unit 311 includes an iris matching unit 3113. The information processing apparatus 3 performs iris authentication. The other features of the information processing apparatus 3 may be the same as the other features of the information processing apparatus 2 according to the second embodiment. Therefore, in the following, only the parts that differ from the previously described embodiments will be described in detail, and the other redundant parts will be omitted as appropriate.

### 3-2: Information Processing Operations Performed by the Information Processing Apparatus 3

Referring to FIG. 3, the information processing operations performed by the information processing apparatus 3 will be described. The information processing apparatus 3 may start the information processing operations in response to the instructions to perform authentication from the target person.

A biometric information acquisition unit 314 acquires eye images including an iris (step S20). The biometric information acquisition unit 314 may acquire the eye images captured by the infrared camera. Alternatively, the biometric information acquisition unit 314 may acquire the eye images captured by a visible light camera. In addition, in case the information processing apparatus 3 performs multimodal authentication including the iris authentication and face authentication, the biometric information acquisition unit 314 may acquire the eye images and face images.

The iris matching unit 3113 performs the iris authentication of the target person using the eye image including the iris of the target person. The iris matching unit 3113 matches the eye image of the target person with the eye images registered in the registered biometric information storing unit 222 (step S21). The iris matching unit 3113 may match features extracted from the eye image of the target person with features registered in the registered biometric information storing unit 222.

An authentication determination unit 3112 determines whether the authentication was successful using the matching results from the iris matching unit 3113 (step S22). In case the authentication is successful (step S22: Yes), a storing control unit 312 stores the target information including authentication information including date and time information indicating date and time when iris authentication using the eye image was performed, and an authentication score of the performed iris authentication, in association with the target person in a target information storing unit 321 (step S23).

An estimation unit 3131 estimates the body information of the target person using two or more pieces of the target information related to the authentication performed at different times of the target person stored in the target information storing unit 321 (step S24). The estimation unit 3131 estimates the body information related to the body of the target person using two or more pieces of the target information related to the iris authentication performed on different dates and times stored in the target information storing unit 321. The estimation unit 3131 may estimate the health condition related to the eyes of the target person. The estimation unit 3131 may estimate the possibility of eye diseases.

### [Estimation Based on Eye Images]

The estimation unit 3131 may estimate the health condition related to the eyes of the target person based on the eye images of the target person. The estimation unit 3131 may estimate the health condition related to the eyes of the target person based on the time series changes in the eye images.

The estimation unit 3131 may detect discoloration of the skin around the eyes from the eye images. The discoloration of the skin may be darkening of the skin. The estimation unit 3131 may detect discoloration of the skin around the eyes in case the color of the skin around the eyes differs from the color of other areas of the skin by a predetermined amount or more. Discoloration of the lower skin around the eyes may be considered dark circles under the eyes, and an output unit 313 may notify the target person of sleep deprivation.

The estimation unit 3131 may detect discoloration of a sclera from the eye images. Discoloration of the sclera may include cases where the sclera includes more blood color than a predetermined amount. As fatigue accumulates, the whites of the eyes become bloodshot, so the estimation unit 3131 may estimate poor body condition in case the bloodshot appearance of the whites of the eyes exceeds a predetermined amount. The estimation unit 3131 may detect overwork. Additionally, the estimation unit 3131 may detect that the sclera is more yellow than the specified color. In this case, the estimation unit 3131 may estimate that internal organs are in poor condition.

The estimation unit 3131 may detect differences between the left and right eyes from the eye images. Differences between the left and right eyes may include differences in eye size. Differences in the eye size may include differences in eyelid opening. The difference between the left and right eyes may be a difference in pupil size. The difference between the left and right eyes may be a difference in color. The difference between the left and right eyes may be a difference in iris size.

The estimation unit 3131 may detect the state of the iris from the eye images. The state of the iris may include the color of the iris. The estimation unit 3131 may detect changes in the color of the iris from the eye images.

### [Estimation Based on Authentication Scores]

The estimation unit 3131 may estimate the health condition related to the eyes of the target person based on the time series changes in the iris authentication scores stored in the target information storing unit 321.

For example, in case the estimation unit 3131 detects that the authentication score is gradually decreasing, it may estimate that the target person may have an eye disease. In many cases, in case a person has the eye disease, it is not possible to acquire the eye images including the iris suitable for the iris authentication compared to in case the person does not have the eye disease. For example, one change that may occur in case a person has the eye disease is drooping eyelids. In this case, the area of the iris included in the eye image may become smaller. Another change that may occur in the event of the eye disease is clouding of the lens. In this case, the color of the iris may change. Therefore, in the event of the eye disease, the authentication score will often be lower than in case the person does not have the disease.

The estimation unit 3131 may also be able to estimate whether the decline in the authentication score is due to illness or aging. For example, the estimation unit 3131 may estimate that there is a possibility of illness in case it detects a rapid decrease in the authentication score. Eye-related illnesses may include eye diseases such as glaucoma and cataracts, as well as brain nerve diseases related to the eyes.

An output determination unit 3132 determines whether to output the determination result to the target person (step S25). In case it is determined that the result should be output (step S25: Yes), the output unit 313 outputs the body information related to the body of the target person (step S26).

### 3-3: Technical Effects of the Information Processing Apparatus 3

The information processing apparatus 3 according to the third embodiment can notify the target person of the health condition related to the eyes by using information used for the iris authentication.

### 4: Fourth Example Embodiment

Next, the fourth embodiment of the information processing apparatus, information processing method, and recording medium will be described. In the following, the fourth embodiment of the information processing apparatus, information processing method, and recording medium will be described using an information processing apparatus 4 to which the fourth embodiment of the information processing apparatus, information processing method, and recording medium is applied.

### 4-1: Configuration of the Information Processing Apparatus 4

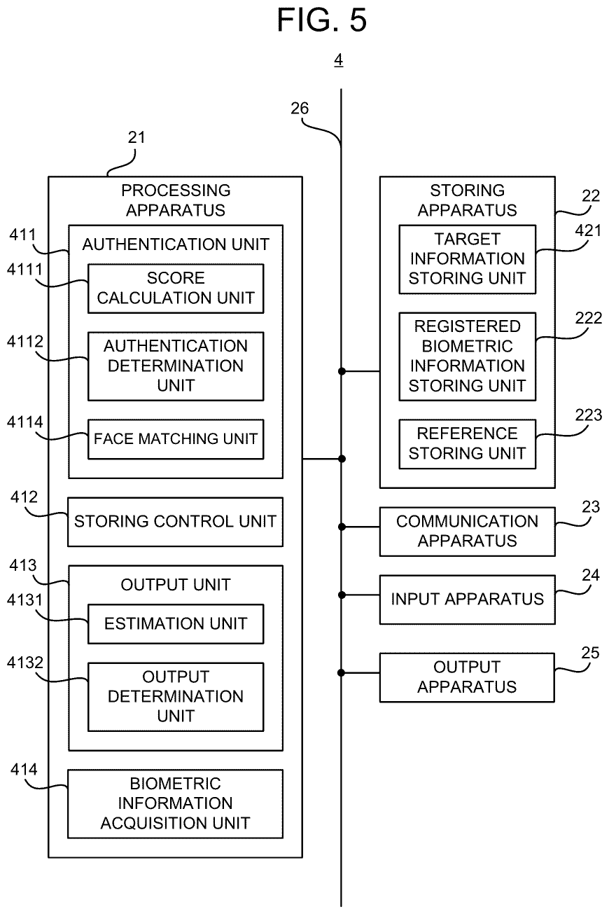

As shown in FIG. 5, the information processing apparatus 4 according to the fourth embodiment is similar to the information processing apparatus 2 according to the second embodiment and the information processing apparatus 3 according to the third embodiment, and includes the processing apparatus 21 and the storing apparatus 22. Furthermore, the information processing apparatus 4 according to the fourth embodiment may also include the communication apparatus 23, the input apparatus 24, and the output apparatus 25, similar to the information processing apparatus 2 according to the second embodiment and the information processing apparatus 3 according to the third embodiment. However, the information processing apparatus 3 may not include at least one of the communication apparatus 23, the input apparatus 24, and the output apparatus 25. The information processing apparatus 4 according to the fourth embodiment differs from the information processing apparatus 2 according to the second embodiment and the information processing apparatus 3 according to the third embodiment in that an authentication unit 411 includes a face matching unit 4114. The information processing apparatus 4 performs face authentication. Other features of the information processing apparatus 4 may be the same as at least one of the other features of the information processing apparatus 2 according to the second embodiment and the information processing apparatus 3 according to the third embodiment. Therefore, in the following, only the parts that differ from the previously described embodiments will be described in detail, and the other redundant parts will be omitted as appropriate.

### 4-2: Information Processing Operations Performed by the Information Processing Apparatus 4

Referring to FIG. 3, the information processing operations performed by the information processing apparatus 4 will be described. The information processing apparatus 4 may start information processing operations in response to the instructions to perform authentication by the target person.

A biometric information acquisition unit 414 acquires face images that include a face (step S20). The biometric information acquisition unit 414 may acquire the face images captured by a visible light camera. Alternatively, the biometric information acquisition unit 414 may acquire the face images captured by an infrared camera. In addition, in case the information processing apparatus 4 performs multimodal authentication including the iris authentication and the face authentication, the biometric information acquisition unit 414 may acquire the eye images and the face images.

The face matching unit 4114 performs the face authentication of the target person using the face image including the face of the target person. The face matching unit 4114 matches the face image of the target person with the face images registered in the registered biometric information storing unit 222 (step S21). The face matching unit 4114 may match features extracted from the face image of the target person with features extracted from the face images registered in the registered biometric information storing unit 222.

An authentication determination unit 4112 determines whether the authentication was successful or not using the matching result acquired by the face matching unit 4114 (step S22). In case the authentication is successful (step S22: Yes), a storing control unit 412 stores the target information, including the face image, the date and time information indicating the date and time when the face authentication using the face image was performed, and the authentication information including the authentication score of the performed face authentication, in a target information storing unit 421 in association with the target person (step S23).

An estimation unit 4131 estimates the body information related to the body of the target person using two or more pieces of the target information related to the face authentication performed on different dates and times stored in the target information storing unit 421 (step S24). The estimation unit 4131 may estimate the health information indicating the health condition as the body information.

### [Estimation Based on Face Images]

The estimation unit 4131 may detect discoloration of the skin of the face from the face image. The discoloration of the skin of the face may be discoloration to brown. In this case, the estimation unit 4131 may estimate the possibility of diseases of organs such as the liver and kidneys.

The estimation unit 4131 may detect differences between the left and right sides of the face from the face image. In case the estimation unit 4131 detects differences between the left and right sides of the face, it may estimate the possibility of diseases related to the brain, including blood vessels, and cranial nerves.

The estimation unit 4131 may estimate the health condition of the target person from the time series changes in the face image.

### [Estimation Based on Authentication Scores]

The estimation unit 4131 may estimate the health condition of the target person based on the time series changes in the authentication scores for the face authentication stored in the target information storing unit 421.

An output determination unit 4132 determines whether or not to output the estimation results (step S25). In case it is determined that the results should be output (step S25: Yes), an output unit 413 outputs the body information related to the body condition of the target person (step S26).

### 4-3: Technical Effects of the Information Processing Apparatus 4

The information processing apparatus 4 according to the fourth embodiment is capable of notifying the target person of the health condition of the target person that can be estimated from the face of the target person using information used for the face authentication.

### 5: Fifth Example Embodiment

Next, the fifth embodiment of the information processing apparatus, information processing method, and recording medium will be described. In the following, the fifth embodiment of the information processing apparatus, information processing method, and recording medium will be described using an information processing apparatus 5 to which the fifth embodiment of the information processing apparatus, information processing method, and recording medium is applied.

### 5-1: Configuration of the Information Processing Apparatus 5

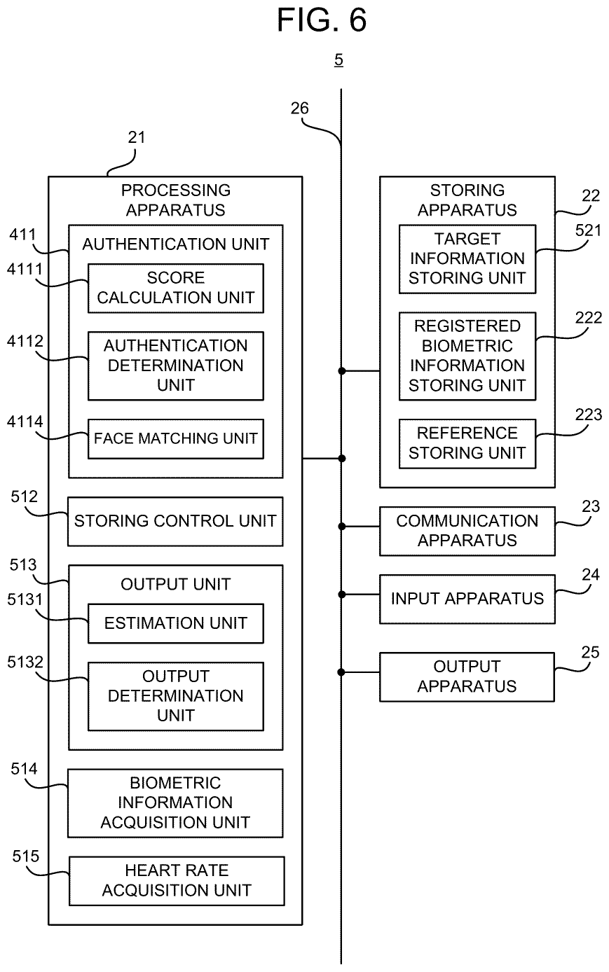

As shown in FIG. 6, the information processing apparatus 5 according to the fifth embodiment is similar to the information processing apparatus 2 according to the second embodiment through the information processing apparatus 4 according to the fourth embodiment, and includes the processing apparatus 21 and the storing apparatus 22. Furthermore, the information processing apparatus 5 according to the fifth embodiment may also include the communication apparatus 23, the input apparatus 24, and the output apparatus 25 in the same manner as the information processing apparatus 2 according to the second embodiment through the information processing apparatus 4 according to the fourth embodiment. However, the information processing apparatus 5 may not include at least one of the communication apparatus 23, the input apparatus 24, and the output apparatus 25. The information processing apparatus 5 according to the fifth embodiment differs from the information processing apparatus 2 according to the second embodiment through the information processing apparatus 4 according to the fourth embodiment in that a heart rate acquisition unit 515 is further realized within the processing apparatus 21. The other features of the information processing apparatus 5 may be the same as at least one of the other features of the information processing apparatus 2 according to the second embodiment through the information processing apparatus 4 according to the fourth embodiment. Therefore, in the following, only the parts that differ from the previously described embodiments will be described in detail, and the other redundant parts will be omitted as appropriate.

### 5-2: Information Processing Operations Performed by the Information Processing Apparatus 5

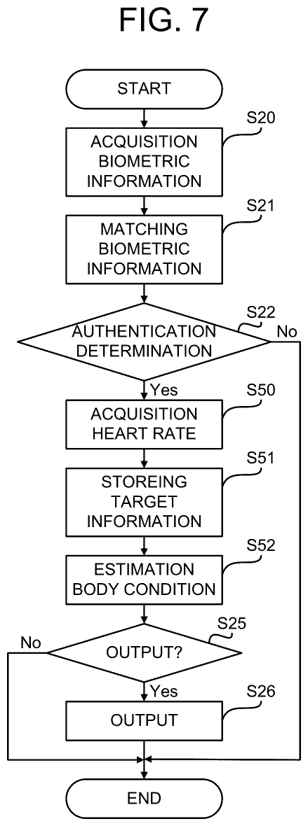

Referring to FIG. 7, the information processing operations performed by the information processing apparatus 5 will be described. FIG. 7 is a flowchart illustrating the flow of information processing operations performed by the information processing apparatus 5. The information processing apparatus 5 may start information processing operations in response to the instructions to perform authentication from the target person.

As shown in FIG. 7, a biometric information acquisition unit 514 acquires the biometric information (step S20). The score calculation unit 2111 matches the biometric information of the target person with the biometric information registered in the registered biometric information storing unit 222 (step S21). The authentication determination unit 2112 determines whether the authentication was successful or not using the matching result from the score calculation unit 2111 (step S22).

In case the authentication is successful in the fifth embodiment (step S22: Yes), the heart rate acquisition unit 515 acquires heart rate information indicating the heart rate of the target person (step S50). The heart rate acquisition unit 515 may acquire the heart rate based on the face image. The heart rate acquisition unit 515 may acquire the heart rate based on the face image captured for the face authentication. That is, the heart rate acquisition unit 515 may acquire the heart rate at the authentication date and time. Alternatively, the heart rate acquisition unit 515 may acquire the heart rate from an apparatus that measured the heart rate of the target person.

A target information storing unit 521 stores the target information, which includes the heart rate information and the date and time information indicating the date and time when the heart rate information was acquired, in the target information storing unit 521 in association with the target person (step S51). An estimation unit 5131 estimates the body information related to the body of the target person using two or more pieces of the target information acquired at different dates and times stored in the target information storing unit 521 (step S52). The estimation unit 5131 may detect heart rate abnormalities. The estimation unit 5131 may detect abnormalities or conditions of the target person using daily time-series changes in the heart rate. The estimation unit 5131 may learn a heart rate of an individual and determine whether it is high or low depending on the individual.

An output determination unit 5132 determines whether to output the estimation result (step S25). In case it is determined that the results should be output (step S25: Yes), an output unit 513 outputs the body information related to the body of the target person (step S26).

### 5-3: Technical Effects of the Information Processing Apparatus 5

The information processing apparatus 5 according to the fifth embodiment can notify the target person the health condition of the target person related to the heart rate by acquiring the heart rate of the target person.

### 6: Sixth Example Embodiment

Next, the sixth embodiment of the information processing apparatus, information processing method, and recording medium will be described. In the following, the sixth embodiment of the information processing apparatus, information processing method, and recording medium will be described using an information processing apparatus 6 to which the sixth embodiment of the information processing apparatus, information processing method, and recording medium is applied.

### 6-1: Configuration of the Information Processing Apparatus 6

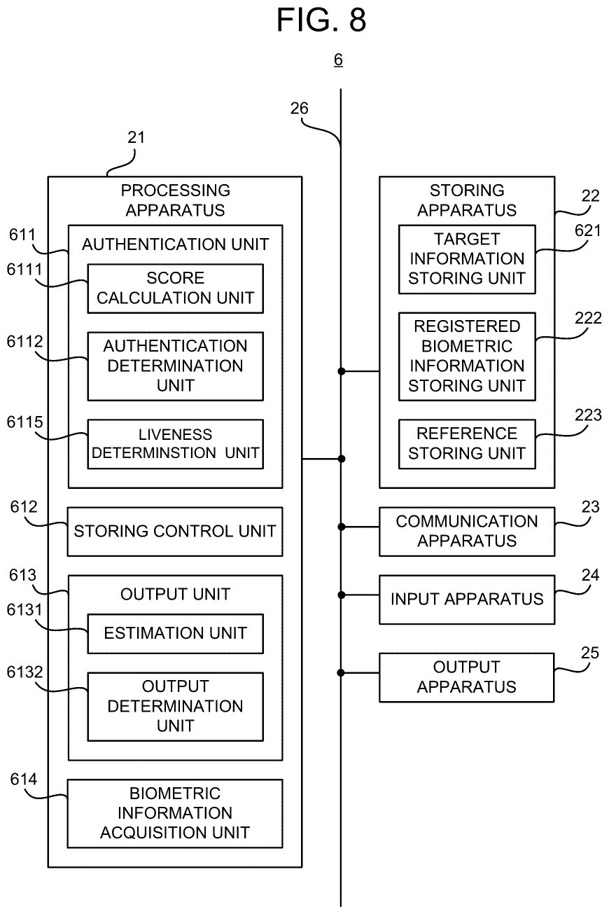

As shown in FIG. 8, the information processing apparatus 6 according to the sixth embodiment is similar to the information processing apparatus 2 according to the second embodiment through the information processing apparatus 5 according to the fifth embodiment, and includes the processing apparatus 21 and the storing apparatus 22. Furthermore, the information processing apparatus 6 according to the sixth embodiment may also include the communication apparatus 23, the input apparatus 24, and the output apparatus 25, similar to the information processing apparatus 2 according to the second embodiment through the information processing apparatus 5 according to the fifth embodiment. However, the information processing apparatus 6 may omit at least one of the communication apparatus 23, the input apparatus 24, and the output apparatus 25. The information processing apparatus 6 according to the sixth embodiment differs from the information processing apparatus 2 according to the second embodiment through the information processing apparatus 5 according to the fifth embodiment in that an authentication unit 611 further includes a liveness determination unit 6115. The other features of the information processing apparatus 6 may be the same as at least one of the other features of the information processing apparatus 2 according to the second embodiment through the information processing apparatus 5 according to the fifth embodiment. Therefore, in the following, only the parts that differ from the previously described embodiments will be described in detail, and the other redundant parts will be omitted as appropriate.

### 6-2: Information Processing Operations Performed by the Information Processing Apparatus 6

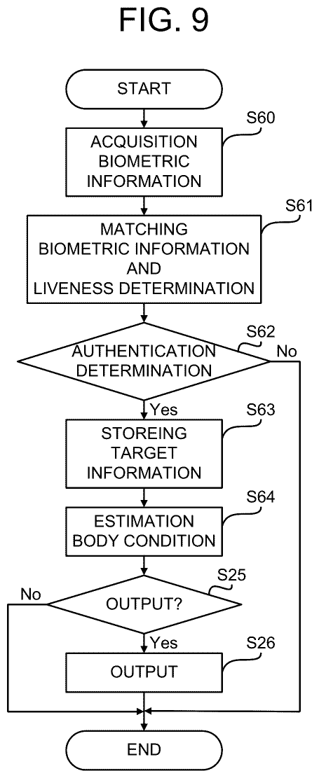

Referring to FIG. 9, the information processing operations performed by the information processing apparatus 6 will be described. FIG. 9 is a flowchart showing the flow of information processing operations performed by the information processing apparatus 6. The information processing apparatus 6 may start information processing operations in response to the instructions to perform authentication from the target person.

As shown in FIG. 9, a biometric information acquisition unit 614 acquires the biometric information (step S60). The biometric information acquisition unit 614 may acquire an image of the target person to be used for matching and body temperature information indicating body temperature of the target person. The biometric information acquisition unit 614 may acquire the image of the target person to be used for matching and the body temperature information indicating the body temperature of the target person at the same time. The information processing apparatus 6 may include a temperature measurement mechanism. For example, a temperature sensor may be provided near the camera.

In the sixth embodiment, the liveness determination unit 6115 performs a liveliness determination. The liveness determination unit 6115 may determine whether the biometric information acquisition unit 614 has acquired an image of a target person that is a living being. The liveness determination unit 6115 may perform the liveliness determination using the body temperature information indicating the body temperature of the target person. In addition, the liveness determination unit 6115 may perform the liveliness determination using the biometric information such as the face images or the eye images acquired by the biometric information acquisition unit 614. Furthermore, the information processing apparatus 6 may determine whether the body temperature of the target person is within a temperature range that permits entry into a restricted area.

A score calculation unit 6111 calculates the authentication score (step S61). The score calculation unit 6111 may calculate the authentication score based on the results of the biometric information matching and the liveliness determination.

An authentication determination unit 6112 determines whether the authentication was successful or not using the authentication score calculated by the score calculation unit 6111 (step S62). In case the authentication is successful (step S62: Yes), a storing control unit 612 stores the target information, including the biometric information and the authentication information related to the authentication using the biometric information, in a target information storing unit 621 in association with the target person (step S63). The storing control unit 612 stores the target information, including the body temperature information and the date and time information indicating the date and time when the body temperature information was acquired, in the target information storing unit 621 in association with the target person.

An estimation unit 6131 estimates the body information related to the body of the target person using two or more pieces of the target information acquired at different times for the target person stored in the target information storing unit 621 (step S64). The estimation unit 6131 may estimate normal body temperature of the target person. The normal body temperature may have a range. For example, the estimation unit 6131 may estimate the normal body temperature of the target person as 36.0 degrees to 37.0 degrees, etc. Normal body temperature varies among individuals. The estimation unit 6131 may learn the normal body temperature that varies among individuals and estimate abnormalities in body temperature according to individual differences. Furthermore, normal body temperature may vary depending on the season, date, time, etc. The estimation unit 6131 may also learn normal body temperature using information such as season and time of day. The estimation unit 6131 may estimate an abnormality in case the body temperature acquired from the target person differs from the normal body temperature by a predetermined amount or more.

An output determination unit 6132 determines whether to output the estimated result (step S25). In case it is determined to output (step S25: Yes), an output unit 613 outputs the body information related to the body of the target person (step S26). The output unit 613 may recommend that the target person visit a medical institution in case the body temperature indicated by the body temperature information is 40 degrees or higher, regardless of whether the normal body temperature of the target person is high or not.

In the sixth embodiment, the liveliness determination using the temperature of the target person was described, but the body temperature may be acquired without performing the liveliness determination. In this case, for example, the biometric information acquisition unit 614 may acquire the body temperature from an apparatus that measures the body temperature of the target person. The biometric information acquisition unit 614 may acquire the body temperature from a wearable apparatus equipped with a body temperature measurement mechanism worn by the target person. In this case, the body temperature may be acquired at predetermined times.

### 6-3: Technical Effects of the Information Processing Apparatus 6

The information processing apparatus 6 according to the sixth embodiment can notify the target person of the health condition of the target person related to body temperature by acquiring the body temperature of the target person.

### 7: Seventh Example Embodiment

Next, the seventh embodiment of the information processing apparatus, information processing method, and recording medium will be described. In the following, the seventh embodiment of the information processing apparatus, information processing method, and recording medium will be described using an information processing apparatus 7 to which the seventh embodiment of the information processing apparatus, information processing method, and recording medium is applied.

### 7-1: Configuration of the Information Processing Apparatus 7

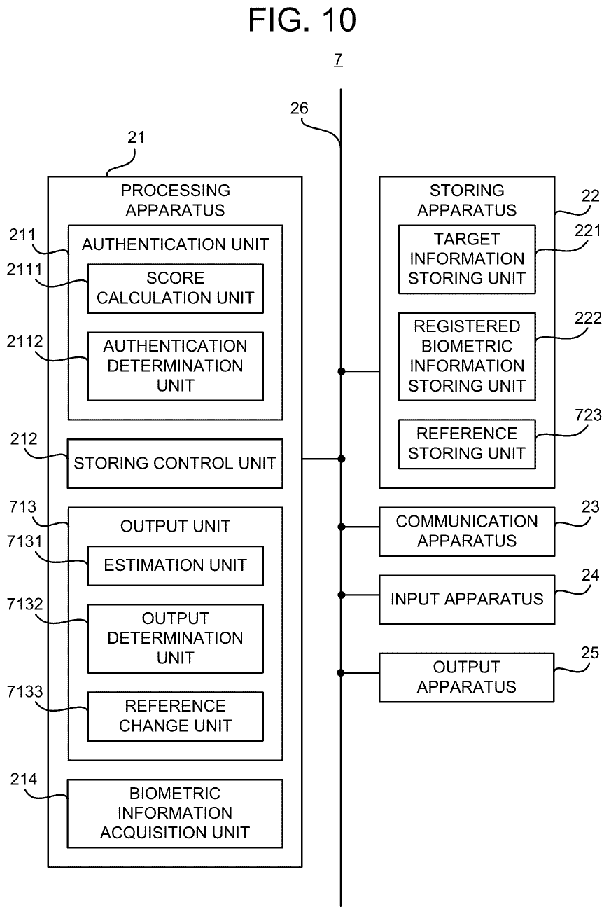

As shown in FIG. 10, the information processing apparatus 7 according to the seventh embodiment is similar to the information processing apparatus 2 according to the second embodiment through the information processing apparatus 6 according to the sixth embodiment, and includes the processing apparatus 21 and the storing apparatus 22. Furthermore, the information processing apparatus 7 according to the seventh embodiment may also include the communication apparatus 23, the input apparatus 24, and the output apparatus 25 in the same manner as the information processing apparatus 2 according to the second embodiment through the information processing apparatus 6 according to the sixth embodiment. However, the information processing apparatus 7 may omit at least one of the communication apparatus 23, the input apparatus 24, and the output apparatus 25. The information processing apparatus 7 according to the seventh embodiment differs from the information processing apparatus 2 according to the second embodiment through the information processing apparatus 6 according to the sixth embodiment in that an output unit 713 further includes a reference change unit 7133. The other features of the information processing apparatus 7 may be the same as at least one of the other features of the information processing apparatus 2 according to the second embodiment through the information processing apparatus 6 according to the sixth embodiment. Therefore, in the following, only the parts that differ from the previously described embodiments will be described in detail, and the other redundant parts will be omitted as appropriate.

### 7-2: Information Processing Operations Performed by the Information Processing Apparatus 7

The output unit 713 outputs the body information related to the body of the target person according to the time period during which the target person was authenticated. The reference change unit 7133 may change the reference information indicating whether or not to output the body information related to the body of the target person according to the time period during which the target person was authenticated. The reference change unit 7133 may change the reference information stored in a reference storing unit 723 according to the time period indicated by the date and time information.

The reference change unit 7133 may change the reference information indicating whether or not to output the body information in case the time period during which the target person was authenticated differs from the usual time period. For example, the reference change unit 7133 may lower a value or range as the reference information for outputting that the target person is in poor body condition in case the target person has been authenticated at midnight a predetermined number of times or more during a predetermined period, thereby making it easier to output that the target person is in the poor body condition. Alternatively, the reference change unit 7133 may raise a value or range as the reference information to avoid overlooking important matters.

In addition, the reference change unit 7133 may change the predetermined threshold value used for comparison with the authentication score used by the authentication determination unit 2112 according to the time period during which the target person was authenticated. For example, since it is often difficult to open one's eyes in the morning, in case using eye images taken in the morning, processing may be performed to supplement for the decrease in the authentication score due to the eyes being closed.

An estimation unit 7131 may determine the activity trends of the target person based on the time when the authentication was performed. In this case, the reference change unit 7133 may change the reference information to determine whether to output the body information based on the activity trends of the target person.

The estimation unit 7131 may estimate whether the target person is in a good sleep state based on the time period in case the target person was authenticated. In addition, the estimation unit 7131 may detect whether the target person has been drinking alcohol based on the chronological changes in the stored images. For example, in the case of authentication in case arriving at work, in case the target person has been drinking alcohol, the administrator may be notified.

### 7-3: Technical Effects of the Information Processing Apparatus 7

The information processing apparatus 7 according to the seventh embodiment can improve user convenience by considering the time of authentication to suppress false rejections at the time of authentication and by changing the reference information that indicates whether to notify the body information.

### 8: Eighth Example Embodiment

Next, the eighth embodiment of the information processing apparatus, information processing method, and recording medium will be described. In the following, the eighth embodiment of the information processing apparatus, information processing method, and recording medium will be described using an information processing apparatus 8 to which the eighth embodiment of the information processing apparatus, information processing method, and recording medium is applied.

### 8-1: Configuration of the Information Processing Apparatus 8

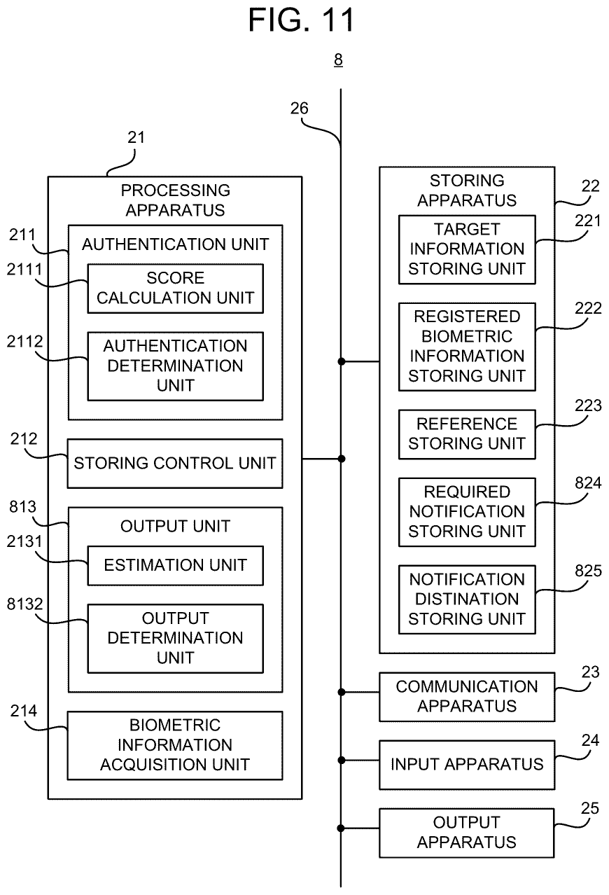

As shown in FIG. 11, the information processing apparatus 8 according to the eighth embodiment is similar to the information processing apparatus 2 according to the second embodiment through the information processing apparatus 7 according to the seventh embodiment, and includes the processing apparatus 21 and the storing apparatus 22. Furthermore, the information processing apparatus 8 according to the eighth embodiment may also include the communication apparatus 23, the input apparatus 24, and the output apparatus 25 in the same manner as the information processing apparatus 2 according to the second embodiment through the information processing apparatus 7 according to the seventh embodiment. However, the information processing apparatus 8 may not include at least one of the communication apparatus 23, the input apparatus 24, and the output apparatus 25. The information processing apparatus 8 according to the eighth embodiment differs from the information processing apparatus 2 according to the second embodiment through the information processing apparatus 7 according to the seventh embodiment in that a required notification storing unit 824 and a notification destination storing unit 825 are further realized within the storing apparatus 22. The other features of the information processing apparatus 8 may be the same as at least one of the other features of the information processing apparatus 2 according to the second embodiment through the information processing apparatus 7 according to the seventh embodiment. Therefore, the following description will focus on the parts that differ from the previously described embodiments, and redundant parts will be omitted as appropriate.

### 8-2: Information Processing Operations Performed by the Information Processing Apparatus 8

Information of recipients who receive the body information of the target person is registered in the notification destination storing unit 825 in association with the target person. The recipients may be person related to the target person. The recipients may be person close to the target person, a manager at a workplace of the target person, etc. The recipients may be designated by the target person. The recipients may be designated based on the application location of the information processing apparatus 8. Contact information of the recipients may be registered in the notification destination storing unit 825 for each target person, as illustrated in the following Table 3. The contact information may be, for example, email addresses, etc.

TABLE 3
[The notification destination storing unit 825]

| Target Person | Recipient | Contact |
|---|---|---|
| P1 | U | P1U |
| P1 | V | P1V |
| P2 | W | P2W |
| P3 | X | P3X |
| P3 | Y | P3Y |
| P3 | Z | P3Z |

.
.
.

In the example shown in Table 3 above, contact information “PIU” for recipient “U” who receives the body information of target person P1, and contact information “PIV” for recipient “V” who receives the body information of target person P1 are registered in the notification destination storing unit 825. Additionally, contact information “P2W” for recipient “W” who receives the body information of target person P2 is registered in the notification destination storing unit 825. Furthermore, contact information “P3X” for recipient “X” who receives the body information of target person P3, contact information “P3Y” for recipient “Y” who receives the body information of target person P3, and contact information “P3Z” for recipient “Z” who receives the body information of target person P3 are registered in the notification destination storing unit 825.

In the eighth embodiment, in step S25 of FIG. 3, an output unit 813 outputs the body information to the recipient who receives the body information of the target person in a specified case. In the required notification storing unit 824, notification content corresponding to each item for each recipient may be stored, as shown in the following Table 4.

TABLE 4
[The required notification storing unit 824]

| Recipient | Item | Notification Content |
|---|---|---|
| U | A | Same as P1 |
| U | B | Notify in case the level is 4 or higher |
| U | C | Notify only the item content and the level |
| U | D | Not required |

.
.
.

In the case illustrated in Table 4 above, in the required notification storing unit 824 stores information indicating that, for item A, the same notification content as that for the target person P1 is to be notified to the recipient “U” who receives the body information of the target person P1. That is, the output unit 813 notifies the recipient “U” in case of notifying the target person P1 for item A. In addition, the required notification storing unit 824 stores information indicating that, for item B, notification shall be made to the recipient “U” in case the level is “4” or higher. In other words, the output unit 813 notifies the recipient “U” in case the level is “4” or higher for item B. Furthermore, the required notification storing unit 824 stores information indicating that, for item C, only the item content and level are to be notified to recipient “U.” That is, the output unit 813 notifies recipient “U” of only the item content and the level for item C. Furthermore, the required notification storing unit 824 stores information indicating that notification is not required for item D to recipient “U.” That is, the output unit 813 does not notify recipient “U” for item D in any case.

### 8-3: Technical Effects of the Information Processing Apparatus 8

The information processing apparatus 8 according to the eighth embodiment can be used for monitoring because it notifies recipients such as the family of the target person of the body information of the target person.

### 9: Ninth Example Embodiment

Next, the ninth embodiment of the information processing apparatus, information processing method, and recording medium will be described. The following describes the ninth embodiment of the information processing apparatus, information processing method, and recording medium using an information processing apparatus 9 to which the ninth embodiment of the information processing apparatus, information processing method, and recording medium is applied.

### 9-1: Configuration of the Information Processing Apparatus 9

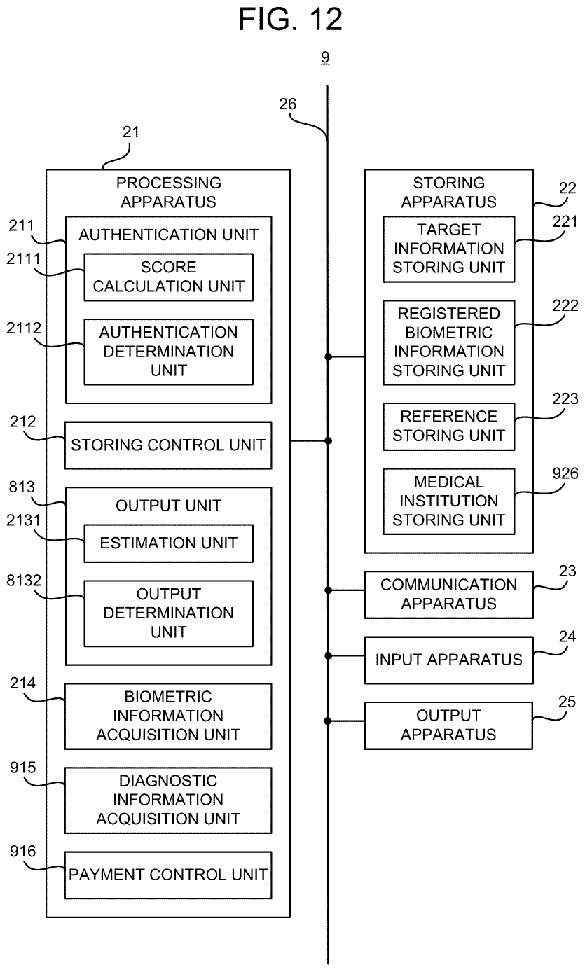

As shown in FIG. 12, the information processing apparatus 9 according to the ninth embodiment is similar to the information processing apparatus 2 according to the second embodiment through the information processing apparatus 8 according to the eighth embodiment, and includes the processing apparatus 21 and the storing apparatus 22. Furthermore, the information processing apparatus 9 according to the ninth embodiment may also include the communication apparatus 23, the input apparatus 24, and the output apparatus 25 in the same manner as the information processing apparatus 2 according to the second embodiment through the information processing apparatus 8 according to the eighth embodiment. However, the information processing apparatus 9 may not include at least one of the communication apparatus 23, the input apparatus 24, and the output apparatus 25. The information processing apparatus 9 according to the ninth embodiment differs from the information processing apparatus 2 according to the second embodiment through the information processing apparatus 8 according to the eighth embodiment in that a diagnostic information acquisition unit 915 and a payment control unit 916 are further realized within the processing apparatus 21, and a medical institution storing unit 926 is further realized within the storing apparatus 22. The other features of the information processing apparatus 9 may be the same as at least one of the other features of the information processing apparatus 2 according to the second embodiment through the information processing apparatus 8 according to the eighth embodiment. Therefore, in the following, only the parts that differ from the previously described embodiments will be described in detail, and the other redundant parts will be omitted as appropriate.

### 9-2: Information Processing Operations Performed by the Information Processing Apparatus 9

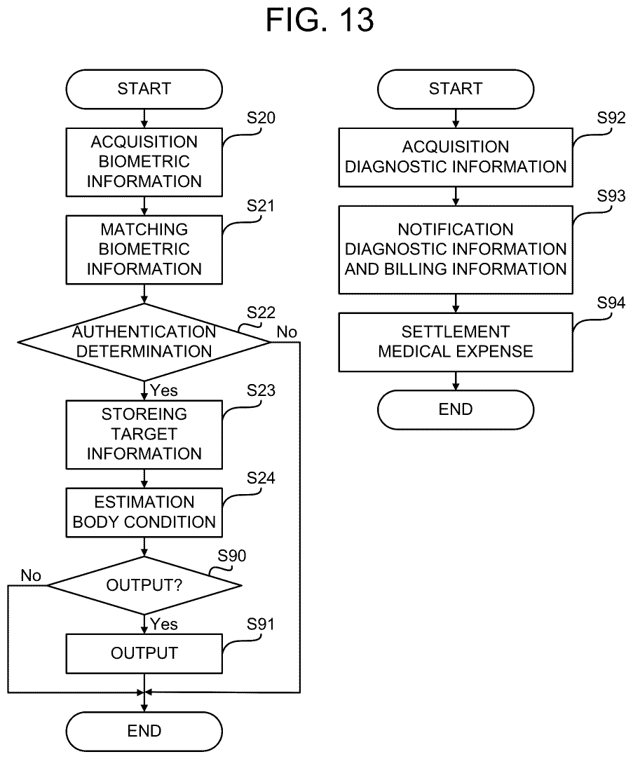

Referring to FIG. 13, the information processing operations performed by the information processing apparatus 9 will be described. FIG. 13 is a flowchart showing the flow of information processing operations performed by the information processing apparatus 9. The information processing apparatus 9 may start information processing operations in response to the instructions to perform authentication from the target person.

### [Notification to Medical Institutions]

As shown in FIG. 13, the biometric information acquisition unit 214 acquires the biometric information (step S20). The score calculation unit 2111 matches the biometric information of the target person with the biometric information registered in the registered biometric information storing unit 222 (step S21). The authentication determination unit 2112 determines whether the authentication was successful based on the matching results acquired by the score calculation unit 2111 (step S22). In case the authentication is successful (step S22: Yes), the storing control unit 212 stores the target information, including the biometric information and the authentication information related to the authentication using the biometric information, in the target information storing unit 221 in association with the target person (step S23). The estimation unit 2131 estimates the body information related to the body of the target person using two or more pieces of the target information related to the authentication performed on different dates and times of the target person stored in the target information storing unit 221 (step S24).

An output determination unit 9132 determines whether it is a predetermined case to output the body information to the medical institution where the target person visits (step S90). The predetermined case may be, for example, in case the level of any item is “4” or higher. The predetermined case may be set by the target person. The predetermined case may be specified by the target person. The predetermined case may be specified by the family of the target person. In case the information processing apparatus 9 is applied to workplace entry/exit of the employee, the specified case may be designated by the workplace. In case the specified case is determined (Step S90: Yes), an output unit 913 outputs the body information to the medical institution where the target person visits. The output unit 913 requests a diagnosis using the body information in case of outputting the body information to the medical institution.

In the medical institution storing unit 926, the target person and the medical institution where the target person visits are registered. The medical institution storing unit 926 may store information of the medical institution responsible for each item for each target person, as shown in Table 5 below.

TABLE 5
[The medical institution storing unit 926]

| Target person | Item | Contact | Withdrawal Account |
|---|---|---|---|
| P1 | A | DA | Account 1 |
| P1 | B | DB | Account 2 |
| P1 | C | DC | Account 1 |
| P1 | D | DD | Account 1 |

.
.
.

In the example shown in Table 5 above, the medical institution storing unit 926 stores “DA” as the contact information for the medical institution responsible for item A for target person P1, “DB” as the contact information for the medical institution responsible for item B, “DC” as the contact information for the medical institution responsible for item C, and “DD” as the contact information for the medical institution responsible for item D.

The output unit 913 may send information used to estimate the body information of the target person to the relevant contact registered in the medical institution storing unit 926. The information used to estimate the body information of the target person may be relevant information, and the output unit 913 may send images of the target person and various numerical logs. The output unit 913 may also provide logs of purchases of the target person at a store, activities at a fitness gym, etc.

[Feedback from the Medical Institutions]

As shown in FIG. 13, the diagnostic information acquisition unit 915 acquires diagnostic information including diagnostic results from the medical institution that received the target information in step S91 (step S92). The diagnostic information may include the contents of the diagnosis made by the medical institution based on the target information regarding the matters estimated by the estimation unit 2131. The diagnostic information may include matters discovered by deriving from the matters estimated by the estimation unit 2131.

The output unit 913 notifies the target person of the diagnostic information and payment information (step S93). In the ninth embodiment, information may be notified to persons other than the target person, as in the eighth embodiment. The output unit 913 may notify the target person and the recipients other than the target person of the diagnostic information and the payment information as appropriate. Additionally, the output unit 913 may, depending on the situation, notify the diagnostic information to the recipients other than the target person without notifying the target person.

The diagnostic information may include a prescription. Alternatively, within the processing apparatus 21, a mechanism may be further implemented to arrange for delivery of medication prescribed by a doctor directly to home of the target person.

Furthermore, in case the doctor determines that a medical consultation is necessary, within the processing apparatus 21, a mechanism for coordinating schedules of the target person and the doctor may be further implemented. Such the medical consultation may be conducted online.

The payment control unit 916 settles medical expenses (step S94). The medical institution storing unit 926 may store, as shown in the example in Table 5 above, a withdrawal account for each item for each target person. In the example shown in Table 5 above, the medical institution storing unit 926 may be registered “Account 1” as a withdrawal account for the item A related to the target person P1, “Account 2” as the withdrawal account for the item B to the target person P1, “Account 1” as the withdrawal account for the item C to the target person P1, and “Account 1” as the withdrawal account for the item D to the target person P1. The payment control unit 916 may settle fee incurred for diagnosis based on the information registered in the medical institution storing unit 926.

Note that the example above illustrates a case where medical institutions and withdrawal accounts can be registered for each item, but it is also possible to register one medical institution and one account for each target person. Additionally, for example, in case the target person is a child, the account of another person, such as guardian etc. of the child, may be registered as the withdrawal account. In this embodiment, direct payment from an account as an example is used, but in addition to or instead of direct payment from an account, card payment using a credit card, etc., may be implemented. Payment using a QR code via a smartphone, etc., may also be implemented.

Furthermore, the medical institution may be determined by an administrator of the information processing apparatus 9. For example, in case the information processing apparatus 9 is applied to an apartment complex, the apartment complex may appoint its own doctor.

### 9-3: Technical Effects of the Information Processing Apparatus 9

The information processing apparatus 9 according to the ninth embodiment enables diagnosis without conducting a medical interview, and in case treatment is possible by prescribing medicine, the target person can automatically receive treatment. The information processing apparatus 9 can contribute to the early detection of diseases.

### 10: Supplementary Note

The following supplementary note is disclosed regarding the embodiments described above.

### Supplementary Note 1

An information processing apparatus including:
an authentication unit that performs an authentication on a target using biometric information of the target;
a storing unit that stores target information in a storing apparatus in association with the target, wherein the target information including the biometric information and authentication information related to the authentication using the biometric information; and
an output unit that outputs body information related to a body of the target, which is estimated using two or more pieces of the target information of the target stored in the storing apparatus.

### Supplementary Note 2

The information processing apparatus according to Supplementary Note 1, wherein
the authentication information includes date and time information indicating date and time when the authentication was performed by the authentication unit, and an authentication score of the authentication, and
the output unit includes an estimation unit that estimates the body information regarding the body of the target using two or more pieces of the target information of the target relating to the authentications performed at different dates and times stored in the storing apparatus.

### Supplementary Note 3

The information processing apparatus according to Supplementary Note 2, wherein
the authentication unit includes an iris authentication unit that performs iris authentication on the target using an eye image including an iris of the target,
the storing unit stores in the storing apparatus the target information including the eye image, date and time information indicating date and time when the iris authentication using the eye image was performed, and the authentication information including an authentication score of the iris authentication in association with the target, and
the estimation unit estimates the body information regarding the body of the target using two or more pieces of the target information of the target relating to the iris authentications performed at different dates and times stored in the storing apparatus.

### Supplementary Note 4

The information processing apparatus according to Supplementary Note 2, wherein
the authentication unit includes a face authentication unit that performs face authentication on the target using a face image including a face of the target,
the storing unit stores in the storing apparatus the target information including the face image, date and time information indicating date and time when the face authentication using the face image was performed, and the authentication information including an authentication score of the face authentication in association with the target, and
the estimation unit estimates the body information regarding the body of the target using two or more pieces of the target information of the target relating to the face authentications performed at different dates and times stored in the storing apparatus.

### Supplementary Note 5

The information processing apparatus according to Supplementary Note 2, further including a heart rate acquisition unit that acquires heart rate information indicating a heart rate of the target,
wherein the storing unit stores in the storing apparatus the target information including the heart rate information and date and time information indicating date and time when the heart rate information was acquired in association with the target, and
the estimation unit estimates the body information regarding the body of the target using two or more pieces of the target information of the target acquired at different dates and times stored in the storing apparatus.

### Supplementary Note 6

The information processing apparatus according to Supplementary Note 2, further including a body temperature acquisition unit that acquires body temperature information indicating a body temperature of the target,
wherein the storing unit stores in the storing apparatus the target information including the body temperature information and date and time information indicating date and time when the body temperature information was acquired in association with the target, and
the estimation unit estimates the body information regarding the body of the target using two or more pieces of the target information of the target acquired at different dates and times stored in the storing apparatus.

### Supplementary Note 7

The information processing apparatus according to Supplementary Note 2, wherein the output unit outputs the body information regarding the body of the target according to the time zone indicated by the date and time information.

### Supplementary Note 8

The information processing apparatus according to Supplementary Note 1 or 2, further including a recipient registration unit registers the target in association with a recipient who receives the body information of the target, and
wherein the output unit outputs the body information to the recipient in a specified case.

### Supplementary Note 9

The information processing apparatus according to Supplementary Note 1 or 2, further including a medical institution registration unit registers the target in association with a medical institution which the target will visit, and
wherein the output unit outputs the body information to the medical institution in a specified case.

### Supplementary Note 10

An information processing method including:
performing an authentication on a target using biometric information of the target;
storing target information in a storing apparatus in association with the target, wherein the target information including the biometric information and authentication information related to the authentication using the biometric information; and
outputting body information related to a body of the target, which is estimated using two or more pieces of the target information of the target stored in the storing apparatus.

### Supplementary Note 11

A recording medium on which a computer program is stored, the computer program being configured to allow a computer to execute an information processing method including:
performing an authentication on a target using biometric information of the target;
storing target information in a storing apparatus in association with the target, wherein the target information including the biometric information and authentication information related to the authentication using the biometric information; and
outputting body information related to a body of the target, which is estimated using two or more pieces of the target information of the target stored in the storing apparatus.

This disclosure may be changed as appropriate within the scope that does not contradict the technical idea that can be read from the claims and the entire description. The information processing apparatus, information processing method, and recording medium with such changes are also included in the technical idea of this disclosure.

### DESCRIPTION OF REFERENCE CODES

1, 2, 3, 4, 5, 6, 7, 8, 9 information processing apparatus
11, 211, 311, 411, 611 authentication unit
221, 321, 421, 521, 621 target information storing unit
13, 213, 313, 413, 513, 613, 713, 813, 913 output unit
2111, 3111, 4111, 6111 score calculation unit
2112, 3112, 4112, 6112 authentication determination unit
12, 212, 312, 412, 512, 612 storing control unit
2131, 3131, 4131, 5131, 6131, 7131 estimation unit
2132, 3132, 4132, 5132, 6132, 7132, 8132, 9132 output determination unit
214, 314, 414, 514, 614 biometric information acquisition unit
3113 iris matching unit
4114 face matching unit
515 heart rate acquisition unit
6115 liveness determination unit
7133 reference change unit
915 diagnostic information acquisition unit
916 payment control unit
222 registered biometric information storing unit
223, 723 reference storing unit
224, 824 required notification storing unit
825 notification destination storing unit
926 medical institution storing unit

What is claimed is:

1. An information processing apparatus comprising:
at least one memory storing instructions; and
at least one processor that is configured to execute the instructions to:
perform an authentication on a target using biometric information of the target;
store target information in the at least one memory-a in association with the target, wherein the target information including the biometric information and authentication information related to the authentication using the biometric information; and
output body information related to a body of the target, which is estimated using two or more pieces of the target information of the target stored in the at least one memory.

2. The information processing apparatus according to claim 1, wherein
the authentication information includes date and time information indicating date and time when the authentication was performed, and an authentication score of the authentication, and
the at least one processor that is configured to execute the instructions to estimate the body information regarding the body of the target using two or more pieces of the target information of the target relating to the authentications performed at different dates and times stored in the at least one memory.

3. The information processing apparatus according to claim 2, wherein the at least one processor that is configured to execute the instructions to:
perform iris authentication on the target using an eye image including an iris of the target:
store in the at least one memory the target information including the eye image, date and time information indicating date and time when the iris authentication using the eye image was performed, and the authentication information including an authentication score of the iris authentication in association with the target; and
estimate the body information regarding the body of the target using two or more pieces of the target information of the target relating to the iris authentications performed at different dates and times stored in the at least one memory.

4. The information processing apparatus according to claim 2, wherein the at least one processor that is configured to execute the instructions to:
perform face authentication on the target using a face image including a face of the target;
store in the at least one memory the target information including the face image, date and time information indicating date and time when the face authentication using the face image was performed, and the authentication information including an authentication score of the face authentication in association with the target; and
estimate the body information regarding the body of the target using two or more pieces of the target information of the target relating to the face authentications performed at different dates and times stored in the at least one memory.

5. The information processing apparatus according to claim 2, wherein the at least one processor that is configured to execute the instructions to:
acquire heart rate information indicating a heart rate of the target;
store in the at least one memory the target information including the heart rate information and date and time information indicating date and time when the heart rate information was acquired in association with the target; and
estimate the body information regarding the body of the target using two or more pieces of the target information of the target acquired at different dates and times stored in the at least one memory.

6. The information processing apparatus according to claim 2, wherein the at least one processor that is configured to execute the instructions to:
acquire body temperature information indicating a body temperature of the target;
store in the at least one memory the target information including the body temperature information and date and time information indicating date and time when the body temperature information was acquired in association with the target; and
estimate the body information regarding the body of the target using two or more pieces of the target information of the target acquired at different dates and times stored in the at least one memory.

7. The information processing apparatus according to claim 2, wherein the at least one processor that is configured to execute the instructions to output the body information regarding the body of the target according to the time zone indicated by the date and time information.

8. The information processing apparatus according to claim 1, wherein the at least one processor that is configured to execute the instructions to:
register the target in association with a recipient who receives the body information of the target; and
output the body information to the recipient in a specified case.

9. The information processing apparatus according to claim 1, wherein the at least one processor that is configured to execute the instructions to:
register the target in association with a medical institution which the target will visit; and
output the body information to the medical institution in a specified case.

10. An information processing method comprising:
performing an authentication on a target using biometric information of the target;
storing target information in at least one memory in association with the target, wherein the target information including the biometric information and authentication information related to the authentication using the biometric information; and
outputting body information related to a body of the target, which is estimated using two pieces or more of the target information of the target stored in the at least one memory.

11. A non-transitory recording medium on which a computer program is stored, the computer program being configured to allow a computer to execute an information processing method comprising:
performing an authentication on a target using biometric information of the target;
storing target information in at least one memory in association with the target, wherein the target information including the biometric information and authentication information related to the authentication using the biometric information; and
outputting body information related to a body of the target, which is estimated using two pieces or more of the target information of the target stored in the at least one memory.
